(19) United States
(12) Patent Application Publication
(10) Pub. No.: US 2018/0035990 A1
(43) Pub. Date: Feb. 8, 2018

(54) SURGICAL RETRACTOR BLADE WITH DISTAL END FORMATION FOR ENGAGING ANCHOR PIN

(71) Applicant: Behzad EFTEKHAR, New South Wales (AU)

(72) Inventor: Behzad EFTEKHAR, New South Wales (AU)

(21) Appl. No.: 15/551,524

(22) PCT Filed: Feb. 16, 2016
(86) PCT No.: PCT/AU2016/000042

§ 371 Date: Aug. 16, 2017

(30) Foreign Application Priority Data
Feb. 16, 2015 (AU) 2015900493

(51) Int. Cl. A61B 17/02

(52) U.S. Cl. CPC A61B 17/02; A61B 17/025; A61B 2017/0256; A61B 2017/00477

(57) ABSTRACT

A retractor blade comprising a blade body having first and second ends, the first, end being a fee end and the second end comprising at least one formation which allows releasable engagement with a bone anchor pin, the second end formation retaining said bone anchor pin such that retention of the anchor pin allows the retractor to move with said anchor pins during distraction of bone m which said anchor pins are inserted.

### BACKGROUND

The present invention relates to soft tissue retractor blades for use in surgery and more particularly relates to a retractor blade have a distal end formation which is capable of releasable engagement with a bone anchor and which thereby moves with the anchor while engaged in retraction and contributes to resist unwanted withdrawal. More particularly, the invention relates to a retracting blade having a distal end geometry which engages a pin anchor locatable in vertebral hone during retraction of soft tissues to enable safer, more convenient and efficient retraction of those tissues. The invention further provides a retraction blade having a formation at a distal end which at least partially engages an anchor pin via a recess which receives and retains the retractor. The invention further provides a retractor blade in which the distal end formation allows retraction of veins and arteries and other soft tissues during spinal surgery while in engagement with a bone pin. Although the invention is to be described below in relation to spinal vertebra the principles and geometry embodied in the retractor blade have applications wherever bone pin anchors or the like are used.

### PRIOR ART

There are in existence retractor blades Which retract soft tissue during spinal surgery. Such retractor blades are maintained at a setting to allow a surgeon access to a spinal disc space and vertebrae in procedures such as cervical and lumbar spinal disc surgery. Retractors are also used in operations requiring distraction of spinal vertebra. The process of axial separating spinal vertebra is termed distraction. This involves insertion of a spreading type instrument into an excised disc space which engages the upper and lower vertebral bodies and when applied separates them. This is known as intervertebral distraction.

Alternatively, in the cervical spine, a procedure known as non-intervertebral distraction may be used in which long screws are inserted into the upper and lower vertebral bodies. Surrounding soft tissues must be held apart by retractors. Once retracted, there is a natural elasticity of the stretched tissues tending to wound closure, so it is essential to employ retractors which effectively restrain soft tissues to allow clear access to a surgical site.

It is important to reduce trauma to soft tissues which may occur from contact with parts of retractor blades so that the surgical procedure is as minimally invasive as possible and thus minimally interferes with and minimally traumatizes the organs, tissues and vasculature being displaced to allow access to the vertebral region being treated. Various known retractor blades each have a different geometry but similar purpose and fundamental form. Each are typified in having means at a first end for engaging a support assembly or being manually held during an operation, a return body portion and a distal or second end which is capable of engaging bone or soft tissue, thereby providing a point of leverage and contributing to retraction and support of the blade during retraction. The design objective with retraction blades are primarily, optimal stability and safe and efficient retraction of soft tissue.

Whatever level of surgery is being performed, there is an initial approach requiring some degree of vascular dissection. The approach and surgery generally requires the use of at least handheld retractors, at least initially, which may then be replaced with fixed retractors to maintain retraction for the rest of the procedure.

Fixed retractors require either internal fixation to spinal vertebra or external fixation using a table mounted system. Retractors are usually positioned to hold tissues away from the surgical field both laterally (side to side) and longitudinally (up and down) relative to a spine. Existing retractors may be internally or externally fixed. Internal fixation of retractors is utilized to hold the left common iliac vein or other tissues in a retracted position. Due to the difficulty and dangers of moving and keeping the blood vessels retracted during some surgeries such as anterior lumbar surgery, stability of the retractors is particularly important. There is a danger that veins may be punctured or squeezed.

The most stable retractors are those embedded in the bone e.g. Steinman pins and Hohmann retractors. Hohmann retractors are conventional retractor blades with a curved pointed end which can be impacted into the bone for stability. Steinman pins are long pins impacted into the bone. Some limited movement of the Hohmann blade is possible by bending. Also known are standard retractor blades that have a channel that allow separate introduction of sharp pins through the channel into the vertebral body thus securing the blade to the spine.

Internally fixed retractors have limitations. Although very stable once in place they are not adjustable and insertion does produce bone injury. Insertion and removal can also be hazardous to vessels or other soft tissues.

There are table based systems, that offer a variety of retractor blades for holding back the tissues connected to a table mounted ring or support frame. Various shapes of blade are available. In order to improve on these and in particular to improve stability of these non internally fixed blades a new shape of retractor blade, called The Brau Blade, was developed. This blade is described in U.S. Pat. No. 6,416,465 which is incorporated by reference herein.

The Brau device is characterised in having a forward directed lip of the distal tip of the blade that curves forward away from the plane of the blade in an opposite direction to that of the handle, i.e towards the spine. This has a gently curved point or blunt lip that contacts the side of the spine and provides more stability than alternative blades during insertion of the retractor and possibly maintenance of retraction. It also has ridges in the lip to increase purchase with the side of the vertebral body or disc.

The lip of the Brau blades and all other conventional blades when used for lateral retraction are in contact with the bone along the side of the body i.e. in contact with the spine along a superior inferior plane. To remain stable they rely on a solid immobile connection to the operating table through various linkages and if in contact with the bone, a force directed along the edge of the blade (the lip) parallel to this radius of the body. This stability is easily achieved when held in the hand, but not when attached to an external frame because of the moment arm from the end of the blade to the fixed anchorage at the side of the table: (see assembly of FIG. 1 below).

Many conventional blades sit beside the bone with a lip curving away from the bone. Contact with the bone provides an additional point of stability. There are disadvantages with retractors which rely on support frames and those that rely on a sharp end or some other termination which engages bone. If a constant force is applied from the frame along the line of the blade, pushing the blade against the spine helps stability, but this can easily lead to the blade slipping or sliding posteriorly and causing tissue injury when the spine moves. Inadvertent downward pressure by surgeons or their assistants on these blades also is a problem as it leads to posterior displacement, because there is little or no support for the blade from the bone relying as it dies essentially on friction grip. This can lead to soft tissue injury in the region in which the surgery is performed.

When retracting longitudinally using retractor blades, the edge of the blades can be pushed down onto the of the vertebral bodies where they are lying across the spine. This gives them much greater relative stability. One of the limitations of existing retraction systems is the tendency for the vessels, to bulge around the retractor, which can expose the vein to injury and impede the surgeon. Excessive retraction can lead to direct vessel injury, bleeding or thrombosis, and irregular retraction. It is desirable to minimize the amount of retraction used and to use smooth retraction over an area, particularly when retracting blood vessels in order to reduce turbulence and thereby the potential for thrombosis within the vessel.

Numerous retractor blades exist for use in surgery of various shapes and geometry. For example one a hand held retractor blade is known with two surfaces at right angles to each other over the length of the blade. The end is shaped for bone contact.

There are other blade assemblies in the prior art such as that disclosed in U.S. Pat. No. 6,692,434 which discloses a method and device for a retractor for microsurgical intermuscular lumbar arthrodesis. One blade is bent at an end portion thereof in a direction away from the handle portion. The other blade has first and second blade faces, with the second face having at least two toothed structures located thereon.

A combined distracter and retractor instrument for use during a spinal surgery procedure is disclosed in US patent application No. 20050154395. The surgical instrument disclosed is configured to distract two adjacent vertebral elements and retract the nerve root to provide access to the distracted site. The instrument includes an elongated blade member having a wing located on an edge of the blade member, and a handle. This instrument is primarily for use in posterior access to the spine. The instrument has some soft tissue retracting capacity but is restricted in its use as it must be held by such persons as assistants and once located between vertebra must be rotated to procure any retraction.

Although there are a variety of retractor blades currently in use, in view of the disadvantages inherent in those blades, there remains room for improvement in the geometry of the blades with the objective of providing a blade which is easy to use, efficiently maintains retraction of soft tissue and does not obstruct the surgeons path to the operating site and does not compromise retraction in the event of unwanted loading such as inadvertent bumping during surgery.

### INVENTION

The present invention provides an alternative to the known retractors seeks to improve the disadvantages of the prior art by providing a retractor blade including a distal end profile which detachably engages an anchor pin.

Outlined broadly below are embodiments and features of the invention to enable the invention to be better understood, and in order that the present contribution to and improvement over the current the art may be better appreciated. There has thus been outlined, rather broadly, the more important features of the invention in order that the detailed description thereof that follows may not only be better understood, but in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways in various rescue circumstances. Also, it is to be understood that the phraseology and terminology employed herein are for the purpose of description and should not be regarded as limiting. As such, those, skilled in the art will appreciate that the conception, upon which this disclosure is based, may readily be utilized as a basis for the designing, of other variations on the assembly, structures, methods and systems for carrying out the purposes of the present invention.

It is therefore an object of the present invention to provide a new and improved device which removes the practical disadvantages encountered using cumin devices and techniques. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The term retraction hen used throughout this description can be taken to mean, displacement and retention of soft tissues which occurs during surgery to assist access to an anatomical region. Throughout the specification, a reference to a retractor blade will include a reference to a device, member arm, plate, rod, restraint or the like which is engaged in the retraction of soft tissues in an anatomical structure of a human or animal.

This retractor according to the invention seeks to improve the stability of retractor blades during spinal surgery by distal end engagement with bone anchor pins. The invention provides an alternative distal end blade shape that improves retraction and resists more effectively any distal end bi directional displacement during surgery which would otherwise result in less efficient retraction compared to existing retractor blades. The formation has the appearance of a toe or tab extending from a location adjacent or near the distal end of the retractor.

In its broadest form the present invention comprises:

a retractor blade comprising a blade body having first and second ends, the first end including means to allow connection of die blade body to a support member or allow manual gripping, the second end comprising at least one formation which allow releasable engagement with a Vertebral bone anchor pin, the second end formation including a recess which receives and retains therein said bone anchor pin such that the retractor is able to move as said anchor pins move dining distraction.

According to a preferred embodiment the second end is distal and comprises a first part disposed in a first orientation and a second part disposed normally to the first part, the second part including an opening which defines an internal space which receives and retains said anchor pin.

In another broad form the present invention comprises:

a surgical retractor blade for retraction of soft tissue during spinal surgery, the blade comprising: a generally elongated planar blade body having first and second ends, the first end providing means for connection of the blade body to a support member or which enables manual gripping by a user, the second end being a distal end comprising at least one formation which extends from the distal end of the blade and which each include a recess which co operatively engage an anchor pin fixed in a spinal vertebrae; wherein the engagement between the pin and formation allows the retractor to move with the anchor pins during distraction of spinal vertebrae and to resist unwanted displacement of the retractor blade during retraction or distraction.

In another broad form the present invention comprises:

retractor blade for retracting soft tissues during spinal surgery, the retractor comprising: a blade body having first and second ends, the first end providing a free non working end and the second end comprising a first part disposed in a first and a second part disposed normally to the first part and including at least one hook like formation which at least partially extends about a vertebral anchor pin sufficient to said orientation allow the retractor to move in co operation with the anchor pm during retraction of vertebral bone.

According to a preferred embodiment the formation includes a side opening which receives the anchor pin and a return which surrounds the pin sufficient to avoid separation between the formation and pin during distraction.

The first part in the first orientation is a transverse distal edge which is part of the blade body. The second part engages the first part via a connection which is preferably integral with the blade body. Preferably, the formation which engages the anchor pins is defined relative to X, Y and Z axes, wherein the X & Y axes are normal to each other and to the Y axis. According to one embodiment, the formation is disposed at or near an outside edge of the blade with the formation extending normally and outwards from a plane of the blade along a Y axis and forms a cantilever. According to one embodiment, the formation engages the retractor blade at a location offset from the longitudinal axis and at a junction between a side and distal edge of the blade. The formation may extend from the blade for a distance within the range of 5 mm-25 mm and an opening to the recess faces in a direction away from the longitudinal axis. Preferably the formation is integral with the blade body. The retractor blade recess has a long dimension along the Y axis normal to a plane of the blade and which is within the range of 5-25 mm. The recess has a short dimension normal to the long dimension and which is within the range of 5 mm to 1.3 mm. The formation preferably has a thickness within the range of 1 mm-8 mm.

In an alternative embodiment the retractor blade body has one or more angled sections which determine the blade body contour and allows the blade to fit soft tissue anatomy. The blade body may assume alternative shapes for strength or orientation as required for a particular anatomy. In a further embodiment the formation which engages the anchor pins comprises a generally L shaped termination at the distal end which is integral with the blade body. This may be formed by an abbreviation of the length of the blade for part of the blade body width. The engaging profile may be pressed from a plane of the blade body to form a tab which is bent to an angle to suit the particular circumstance of pin engagement. Recessed may be pressed into the tab prior to bending.

In another broad form the present invention comprises:

a retractor blade for retracting soft tissues during spinal surgery, the retractor comprising: a blade body having first and second ends, the first end providing means to allow connection of the blade body to a support member or to allow manual gripping, the second end comprising a cantilevered formation extending away from the blade body and including at least one recess which is capable of receiving and retaining a bone anchor pin such that the formation co operates with the pin to restrain unwanted withdrawal of the retractor and allowing the retractor to move in unison with anchor pins during distraction of spinal vertebra.

According to an alternative embodiment, the formation at the working end of the retractor blade is formed in and is integral with the body of the blade and formed by a return portion at the distal end of the blade. In another embodiment, the formation is releasably connected to the blade allowing an operator to select working formations according to surgical requirements. Preferably, the body of the blade is manufactured from a substantially planar plate which is angled or curved to accommodate anatomy in a particular surgical site.

The present invention provides an alternative to the known prior art and the shortcomings identified. The foregoing and other objects and advantages will appear from the description to follow. In the description reference is made to the accompanying representations, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying illustrations, like reference characters designate the same or similar parts throughout the several views. The following detailed description is, therefore, not to be taken in a limiting sense, and the scope of the present invention is best defined by the appended claims. It will be convenient to hereinafter describe the invention in relation to a metal blade in the present exemplary application, but it is to be appreciated that the invention may be manufactured from other materials or combination of materials.

### BRIEF DESCRIPTION OF THE DRAWINGS

The present invention will now be described in more detail according to a preferred embodiment but non limiting embodiment and with reference to the accompanying illustrations, wherein:

FIG. 1 shows a perspective view of a retractor blade according to a preferred embodiment in a first orientation.

FIG. 2 shows a perspective view of a retractor blade according to a preferred embodiment in a second orientation.

FIG. 3 shows a front elevation view of a retractor blade engaging an anchor pin in a spinal vertebrae.

FIG. 4 shows a perspective view of a retractor blade according to an alternative embodiment.

FIG. 5 shows a side elevation view of the retractor of FIG. 4.

FIG. 6 shows a plan view of the retractor of FIG. 4.

FIG. 7 shows a front view of the retractor blade of FIG. 4.

FIG. 8 shows an enlarged but abbreviated side elevation view of the distal end of the blade of FIG. 4.

FIG. 9 shows a perspective view of a retractor blade according to an alternative embodiment.

FIG. 10 shows a plan view of the retractor blade of FIG. 9.

FIG. 11 shows an enlarged view of the distal end formation (circled A in FIG. 10) according to a preferred embodiment.

FIG. 12 shows a cross sectional elevation through a spine having pins connected in vertebra and opposing retractors.

FIG. 13 shows a plan view of the arrangement of FIG. 12 showing a connection regime for two retractors according to one embodiment.

FIG. 14 a,b,c show a schematic view of various angulations which may be applied to retractors described herein to accommodate various anatomical structures

### DETAILED DESCRIPTION

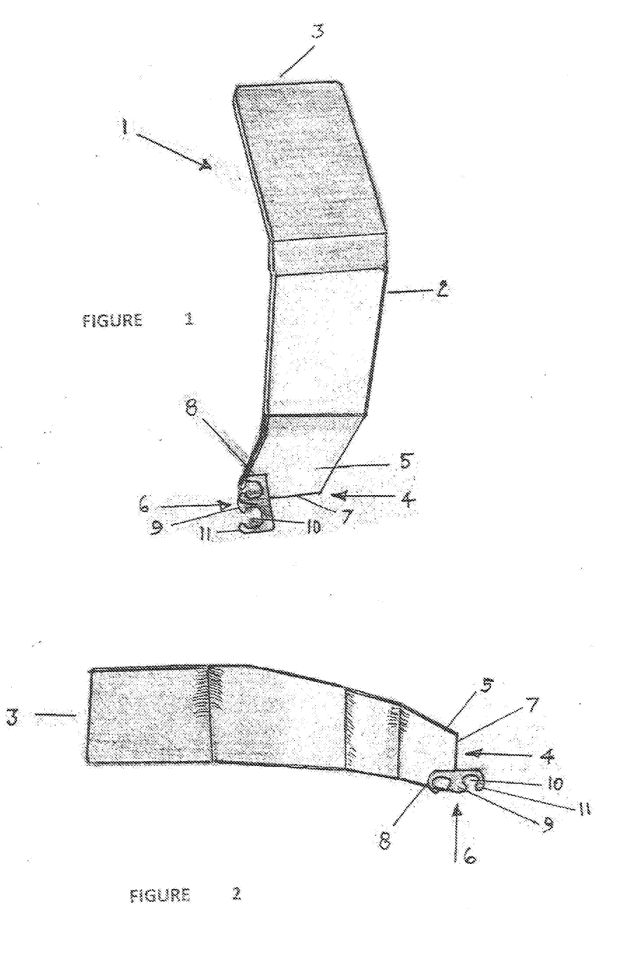

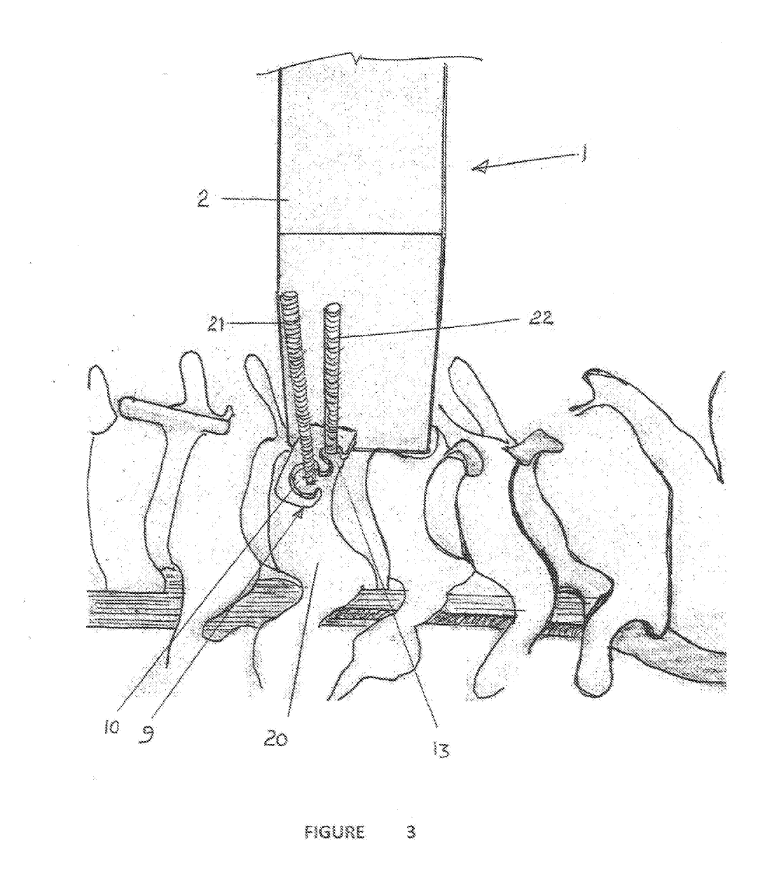

Referring to FIG. 1 there is shown a perspective view of a retractor blade according to a preferred embodiment in a first orientation. Retractor blade 1 comprises a generally elongated blade body 2 having a first free end 3 and second working end 4. First end 3 either provides means for connection to a support (not shown) or is simply adapted for manual operation. Second end 4 comprises a first part 5 oriented in a first direction and a second part 6 connected to the first part and disposed preferably normally to the first part. In a general sense the second part 6 is preferably normal to the orientation of the first part 5 but alternative embodiments are feasible. For instance the second part 6 may be disposed at an angle relative to either a horizontal or vertical angle to the first part 5. The first part 5 terminates in an edge 7 and at or adjacent to edge 7 is at least part of edge 7 which incorporates a connection 8 to formation 9 at working end 4. Formation 9 includes a female recess 10 defined by a hook like profile 11 which receives and retains therein an anchor pin (see FIG. 3 below).

Formation 9 in FIG. 1 is shown connected to part of edge 7. This may be formed in manufacture by a continuation of first part 5 by pressing and forming of the required profile in formation 9 followed by bending to a suitable angle as dictated by the anatomical environment in which the blade is used. It is preferred that formation 9 is integral with part 5 but it will be appreciated by persons skilled in the art that other forms of connection are feasible. In an alternative embodiment, formation 9 may be detachable from part 5 and a variety of sizes and configurations are envisaged.

Edge 7 is when blade 1 is in use, disposed in an orientation which is essentially parallel to a longitudinal axis of a spine—see FIG. 3. The formation 9 extends from first part 5 and may be configured to suit a particular geometry according to the relationship between the edge 7 of blade 1 and the angulation required to enable formation 9 to engage an anchor pin. For example the angle of repose between the formation 9 and the blade body 2 may be an acute or obtuse angle or normal to the blade 1. The angle of repose of formation 9 relative to end 4 dictates the approach angle to the anchor pins. Although one recess is preferred, formation 9 may comprise more than one recess which allows a user to select positioning of the retractor to align with different slots with a position of an anchor pin to which it will be connected.

FIG. 2 shows with corresponding numbering a perspective view of a retractor blade according to a preferred embodiment in a second orientation.

FIG. 3 shows with corresponding numbering a front elevation view of the retractor blade 1 engaging an anchor pin 21 in a spinal vertebrae 20. Formation 9 allows pin 21 to engage recess 10. It will be appreciated by persons skilled in the art that formation 9 may have alternate geometry to allows alternative means for engagement with pins 21 and 22. For instance, the location and a recess or recesses in formation 9 can be arranged to alter the approach direction from which the retractor blade may engage the pin 21. This may be achieved according to one embodiment by alteration of the position of the recesses in the formation 9. In one embodiment the recess can be formed by bifurcated arms extending from the blade body.

An advantage of the blade according to the present invention is that the co operation between the anchor pins and the distal end formation 9 of the blade 1 prevents unwanted slippage or withdrawal of the retractor blade. Since the risk of unwanted displacement or slippage of the retractor is removed, there is less risk of soft tissue injury due to blade displacement during retraction of vertebral distraction.

The distal end formations described herein according to various embodiments may be adapted to known retractor blades or manufactured such that the formation is integral with the blade body. The size and geometry of the blade and profile of the formations is variable according to surgical requirements. For instance a smaller sized retractor would be used for smaller patients and larger proportionate sizes for larger patients.

FIG. 4 shows a perspective view of a retractor blade 30 according to an alternative embodiment. Retractor blade 30 comprises a generally elongated but arcuate blade body 31 having a proximal free end 32 and distal working end 33. Proximal end 32 either provides means for connection to a support not shown) or is simply adapted for manual operation. Distal end 33 comprises a first part 34 oriented in a first direction and a second part 35 integral with the first part and forming a radiused curve which is intended to accommodate contours of patient anatomy. Third part 36 is flattened out and is disposed at 90 degrees to the first part 34. This geometry is illustrative and is not to be taken as limiting of the various geometries which may be selected to accommodate patient anatomies.

The first part terminates in an edge 37 and at or adjacent to edge 37 along at least part of edge 37 is a hook formation 38 which includes a female recess 39 which receives and retains therein an anchor pin (see FIG. 3). Formation 38 is shown integrally connected at one side of retractor blade 30 to part of edge 37. This arrangement may be formed in manufacture by a continuation of first part 33 by pressing and forming of the required profile in formation 38 followed by bending to a suitable angle as dictated by the anatomical environment in which the blade is used. It is preferred that formation 38 is integral with first part 33 but it will be appreciated by persons skilled in the art that other forms of connection are feasible, in an alternative embodiment, formation 38 may be detachable from edge 37 and a variety of sizes and configurations are envisaged.

Edge 37 is when blade 30 1 is in use, disposed in an orientation which is essentially parallel to a longitudinal axis of a spine—see FIGS. 3. The formation 38 is cantilevered from first part 33 and may be configured to suit a particular geometry according to the relationship between the edge 37 of blade 30 and the angulation required to enable formation 38 to engage an anchor pin. For example the angle of repose between the formation 38 and the blade body 31 may be an acute or obtuse angle or normal to the first part 33 of blade 30. The angle of repose of formation 38 relative to the plane of first part 33 dictates the approach angle to the anchor pins. In use when the blade 30 is retracting soft tissue, a distraction force is applied in the direction of arrow 40 to vertebrae via anchor pins, blade 30 is urged in the same direction.

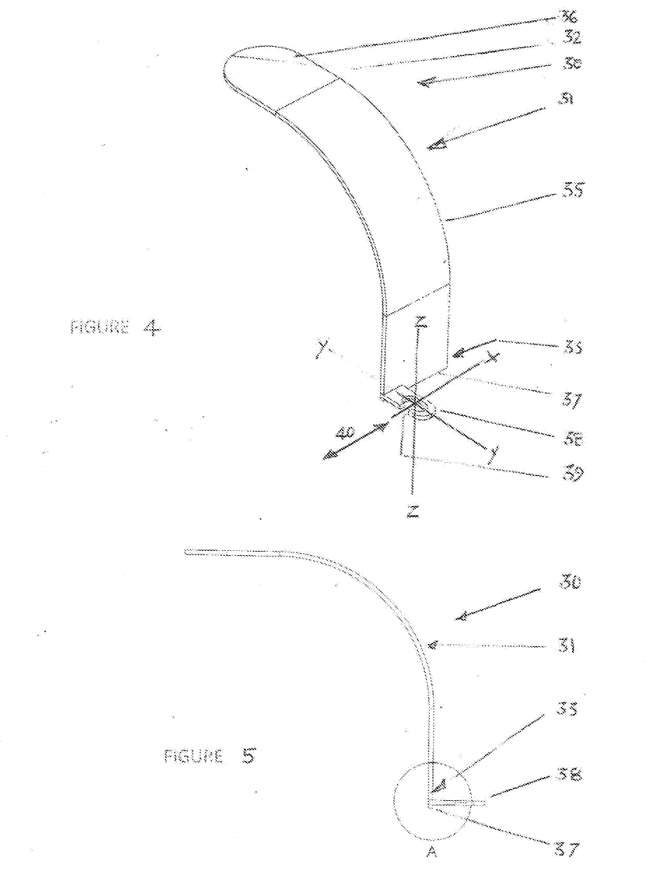

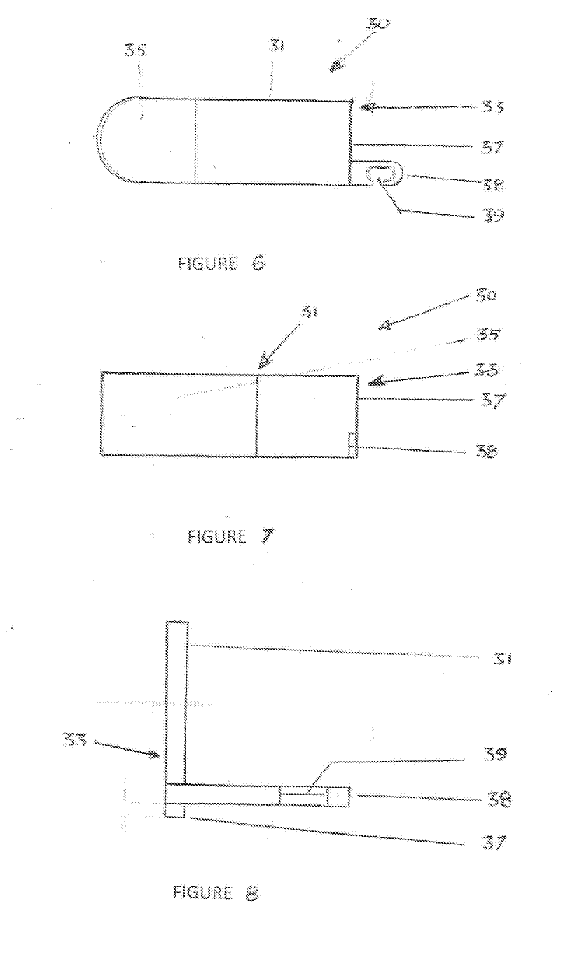

FIG. 5 shows with corresponding numbering a side elevation view of the retractor of FIG. 4. FIG. 6 shows with corresponding numbering a plan view of the retractor of FIG. 4. FIG. 7 shows with corresponding numbering a front view of the retractor blade of FIG. 4. FIG. 8 shows with corresponding numbering an enlarged but abbreviated side elevation view of the distal end of the blade of FIG. 4.

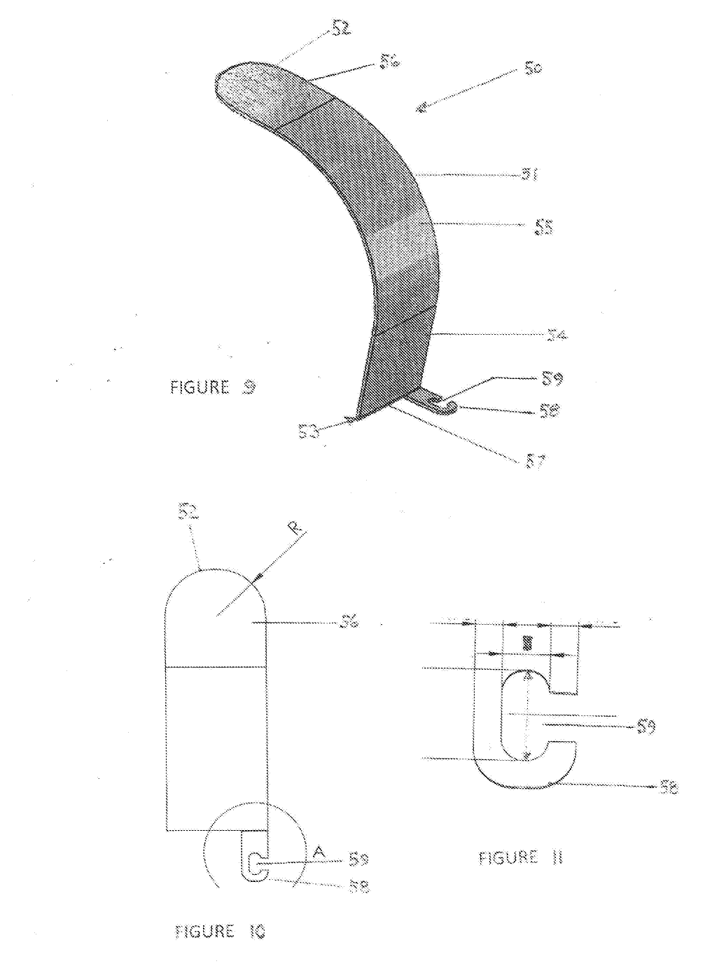

FIG. 9 shows a perspective view of a retractor blade according to an alternative embodiment. Retractor blade 50 is largely the same as retractor blade 30 and comprises a generally elongated but arcuate blade body 51 having a proximal free end 52 and distal working end 53. Proximal end 52 either provides means for connection to a support (not shown) or is simply adapted for manual operation. Distal end 53 comprises a first part 54 oriented in a first direction and a second part 55 integral with the first part and forming a radiused curve which is intended to accommodate contours of patient anatomy. Third part 56 is flattened out and is disposed at 90 degrees to the first part 54. His geometry is illustrative and is not to be taken as limiting of the various geometries which may be selected to accommodate patient anatomies. The first part 53 terminates in an edge 57 and at or adjacent to edge 57 along at least part of edge 57 is a hook formation 58 which includes a female recess 59 which receives and retains therein an anchor pin (see FIG. 3). Formation 38 is shown integrally connected to an opposite side of part of edge 57.

Thus a retractor blade having the hook formation 38 on retractor 30 is a side by side mirror image of the hook formation 58 of the retractor blade 50. This opposite geometry of each formation allows two retractor blades to work side by side and move in opposite direction (away from each other) during distraction of spinal vertebra. Distraction forces are transmitted in anchor pins to the retractor blades causing the blades to move apart ensuring a clear field of view and access for the surgeon. Alternatively, opposing blades may operate on opposite sides of a wound

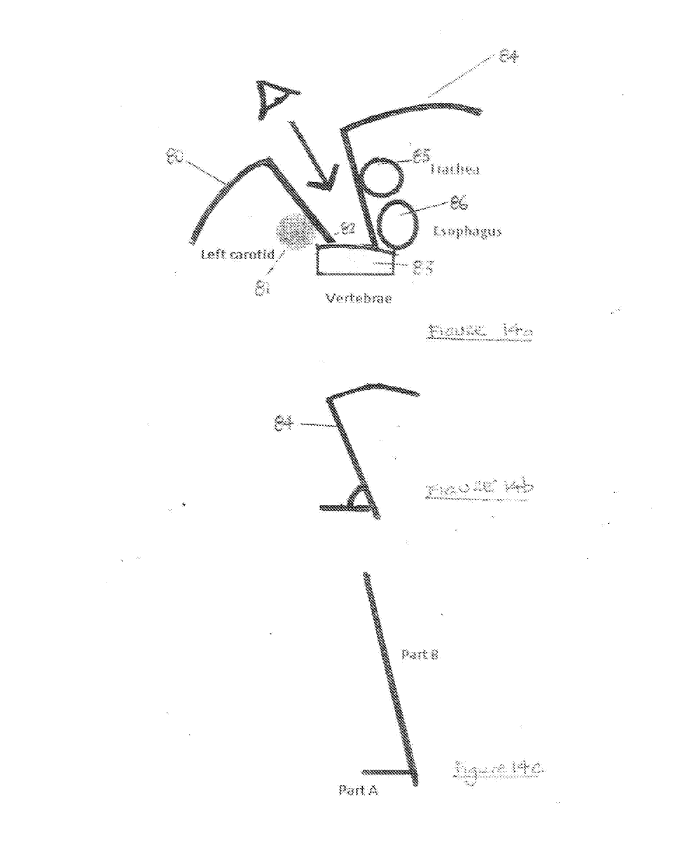

Edge 57 is when blade 50 is in use, disposed in an orientation which is essentially parallel to a longitudinal axis of a spine—see FIG. 13 below. The formation 58 is cantilevered from first part 53 and may be configured to suit a particular geometry according to the relationship between the edge 57 of blade 50 and the angulation required to enable formation 58 to engage an anchor pin. For example the angle of repose between the formation 58 and the blade body 51 may be an acute or obtuse angle or normal to the first part 53 of blade 50. The angle of repose of formation 58 relative to the plane of first part 53 dictates the approach angle to the anchor pins. Such angles are best shown in schematic FIG. 14a below.

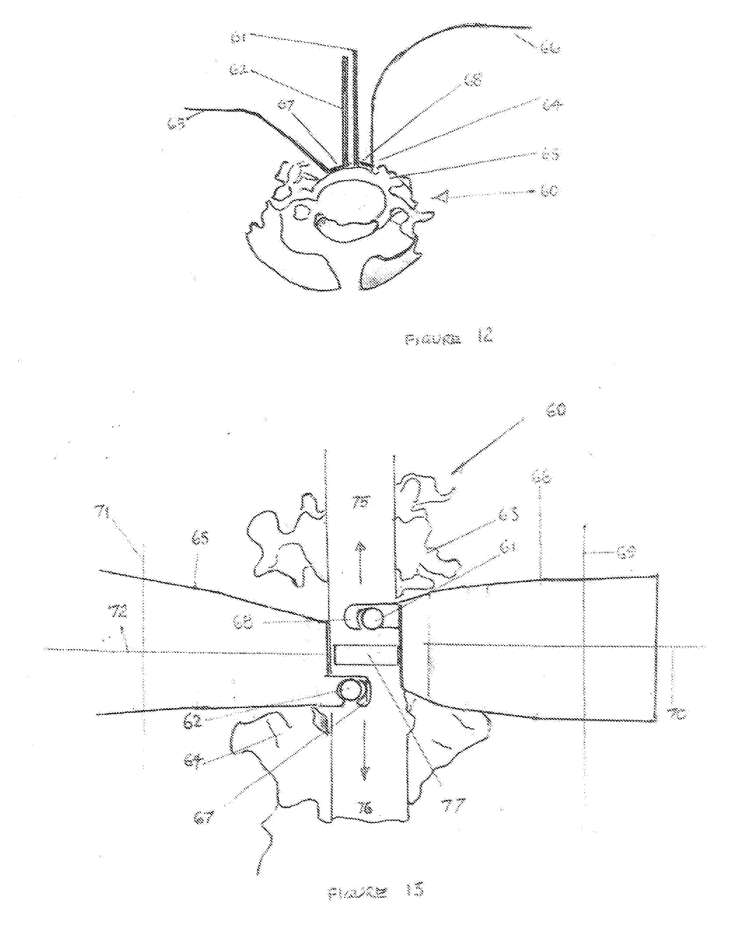

FIG. 10 shows a plan view of the retractor blade of FIG. 9. FIG. 11 shows an enlarged view of circled area A of FIG. 10 comprising the distal end formation according to a preferred embodiment.

FIG. 12 shows a cross sectional elevation through a spine 60 having pins 61 and 62 connected in separate vertebra 63 and 64 (obscured) and opposing retractors 65 and 66. Retractor 65 engages pin 62 via formation 67. Angled retractor 65 has a different profile from curved retractor 66 to accommodate different anatomy. Retractor 66 engages pin 61 via formation 68. It will be appreciated that the side profile of the retractor can be adjusted as required for particular anatomy. FIG. 13 shows with corresponding numbering a plan view of the arrangement of FIG. 12.

From this view its can be seen that formation 67 engages pin 62 and formation 68 engages pin 61. Retractor blade 65 has a long axis 72 and a short transverse axis 71. Likewise retractor 66 has a long axis 70 and transverse axis 69Pins 61 and 62 apply distraction forces in the directions of arrows 75 and 76. This allows access to disc space 77. As pins 61 and 62 distract, retractor blades 65 and 66 can displace along with the pins. FIG. 13 shows one possible geometry for each formation 67 and 68. It will be appreciated that the geometry may be changed and the shape of the formation may also change from that depicted as long as the formations allow the retractors to move with the anchor pins 61 and 62.

FIG. 14 a,b,c show a schematic view of various angulations which may be applied to retractors described herein to accommodate various anatomical structures. FIG. 14 a is a view through a body looking from the head end to the foot end and shows a retractor 80 adjacent the left carotid artery 81 and engaging at its distal end 82 a spinal bone 83. Retractor 84 has an alternative shape and in this case abuts the Trachea 85 and Oesophagus 86. Each end of the retractor blades 80 and 84 engages respective pins which are not shown inserted into spine 83. FIG. 14b shows retractor 84 isolated from its in situ position shown in FIG. 14a.

Angulation is to minimize the retraction and to optimize the vision for a surgeon which on one side is less than 90 degrees and on the other side more than 90 degrees currently one side 85 degrees and other side 140 degrees. Preferably the range is between 20-170 degrees. In FIG. 14c Part A and part B are identified Part B length depends on the depth of the field The curve at the end is to facilitate handling. The width of the part B is to optimise the width of the operating field. Expose the disc space and minimize the force to rotate the retractor the wider the retractor the higher the force by esophagus muscles to rotate the retractor around the pin the range of part B is around 5-50 mm wide.

Part A length should minimize the retraction optimize the width of the disc exposure enough for discectomy aced disc replacement (cages, artificial discs) the range of part A is 5-30 mm; there is a potential to be used for other spinal levels like thoracic, lumbar, other animals). Part A may be attached to part B at different points longitudinally (0-50 mm). Part A may be curved or straight with the width of part A configured to fit an anchor pin, provide enough strength and maximize the operating field. Part A may be inverted (opening inside rather than outside and should be on the either end to maximize the useable width of the retractor.

Various materials may be used for the retractor including metals and plastics and malleable and radiolucent materials. Various sizes and shapes and configurations of a distal end are envisaged which are each able to engage an anchor pin so that the retractor is able to move longitudinally relative a spinal axis.

Variations in the retractor may be made to accommodate different variation in engagement to vertebrae. For instance the length, width and relative heights of horizontal and vertical sections, may vary to accommodate, different locations in the spine and different types of procedures.

It will be further recognised by persons skilled in the art that numerous variations and modifications may be made to the invention without departing from the overall spirit and scope of the invention broadly described herein. Such modifications would allow adaptation of key concepts to provide locking of distraction devices for use in anterior or posterior spinal surgery throughout the length of a spine or in orthopaedics or other surgical disciplines where bony fixation is available.

1. A retractor blade comprising a blade body having first and second ends, the first end being a free end and the second end comprising at least one formation which allows releasable engagement with a bone anchor pin, the second end formation retaining said bone anchor pin such that retention of the anchor pin allows the retractor to move with said anchor pins during distraction of bone in which said anchor pins are inserted.

2. A retractor blade according to claim 1 wherein the formation includes a recess which receives and retains the anchor pin.

3. A retractor blade according to claim 2 wherein the first free end is proximal and the second end is distal.

4. A retractor blade according to claim 3 wherein the retractor blade body is planar.

5. A retractor blade according to claim 4 wherein the distal end formation extends normally from the retractor blade and terminates in a free end.

6. A retractor blade according to claim 5 wherein the retractor blade body has a longitudinal axis, a transverse axis and a bend or radius of curvature relative to the transverse axis.

7. A retractor blade according to claim 6 wherein the retractor blade body has a radius of curvature about the transverse axis for at least part of the length of the blade

8. A retractor blade according to claim 7 wherein the distal end of the retractor blade has an edge from which said formation extends.

9. A retractor blade according to claim wherein the formation extends normally from the distal edge of the blade.

10. A retractor blade according to claim 9 wherein the formation defines an internal space which receives and retains said anchor pin.

11. A retractor blade according to claim 10 wherein the recess in the formation is configured to accommodate an external shape of an anchor pin.

12. A retractor blade according to claim 11 wherein the formation which engages the anchor pins is defined relative to X, Y and Z axes, wherein the X & Y axes are normal to each other and to the Y axis.

13. A retractor blade according to claim 12 wherein the formation is disposed at or near an outside edge of the blade.

14. A retractor blade according to claim 13 wherein the formation extends normally and outwards from a plane of the blade along a Y axis and forms a cantilever.

15. A retractor blade according to claim 14 wherein the formation engages the retractor blade at a location offset from the longitudinal axis.

16. A retractor blade according to claim 15 wherein the formation disposed at a junction between a side and distal edge of the blade.

17. A retractor blade according to claim 16 wherein the formation extends from the blade for a distance within the range of 5 mm-25 mm.

18. A retractor blade according to claim 17 wherein the formation is disposed so that an opening to the recess faces a direction away from the longitudinal axis.

19. A retractor blade according to claim 18 wherein the formation is integral with the blade body.

20. A retractor blade according to claim 19 wherein the recess has a long dimension along the Y axis normal to a plane of the blade and which is within the range of 3 mm to 13 mm.

21. A retractor blade according to claim 20 wherein the recess has a short dimension normal to the long dimension and which is within the range of 2 mm to 13 mm.

22. A retractor blade according to claim 21 wherein the formation has a thickness within the range of 1 mm 8 mm.

23. A retractor blade according to claim 22 wherein the recess is substantially C shaped.

24. A retractor blade according to claim 22 wherein the recess is polygonal.

25. A retractor blade according to claim 22 wherein the recess is circular.

26. A retractor blade according to claim 22 wherein the recess is square.

27. A retractor blade according to claim any one of the forgoing claims wherein the proximal end is connectable to a support member.

28. A retractor blade according to any one of the foregoing claims manufactured from a material selected from titanium stainless steel, chrome cobalt, or plastics.

29. A surgical retractor blade for retraction of soft tissue during spinal surgery, the blade comprising: a generally elongated planar blade body having proximal and distal ends, the proximal end including means for connection of the blade body to a support member or which enables manual gripping by a user, the distal end comprising at least one working formation which extends from the distal end of the blade and which co operatively engages an anchor pin fixed to a spinal vertebrae; wherein the engagement between the pin and said at least one working formation allows the retractor to move with the anchor pins during distraction of spinal vertebrae and to resist any unwanted displacement of the retractor blade during retraction or distraction.

30. A retractor blade for retracting soft tissues during spinal surgery, the retractor comprising: a blade body having first and second ends, the first end providing a free non working end and the second end comprising a first part disposed in a first orientation and a second part disposed normally to the first part and including at least one hook like formation which at least partially extends about a vertebral anchor pin sufficient to said orientation allow the retractor to move in co operation with the anchor pin during retraction of vertebral bone.

31. A retractor blade according to claim 30 wherein the formation includes a side opening which receives the anchor pin and a return which surrounds the pin sufficient to avoid separation between the formation and pin during distraction.

32. A retractor blade according to claim 31 wherein the retractor blade body has one or more angled or curved sections which determine the blade body contour and allows the blade to fit soft tissue anatomy.

33. A retractor blade according to claim 32 wherein the blade body assumes alternative geometry for strength and/or orientation as required for a particular patient anatomy.

34. A retractor blade according to claim 33 wherein the formation which engages the anchor pins comprises a generally C shaped recess and is integral with the blade body.

35. A retractor blade according to claim 34 wherein the formation is pressed from a plane of the blade body to form a tab which is bent to an angle to suit the particular circumstance of pin engagement.

36. A retractor blade according to claim 35 wherein the recess of the formation is pressed into the tab prior to bending.

37. A retractor blade for retracting soft tissues during spinal surgery, the retractor comprising: a blade body having first and second ends, the first end providing means to allow connection of the blade body to a support member or to allow manual gripping, the second end comprising a cantilevered formation extending distally from the blade body and including at least one recess which is capable of receiving and retaining a bone anchor pin such that the formation co operates with the pin to restrain unwanted withdrawal of the retractor and allowing the retractor to move in unison with anchor pins during distraction of spinal vertebra.

38. A retractor blade according to claim 37 wherein the body of the blade is manufactured from a substantially planar plate which is angled or curved to accommodate anatomy in a particular surgical site.

39. A retractor blade according to claim 38 wherein the formation at the working end of the retractor blade is formed in and is integral with the body of the blade and formed by a return portion at the distal end of the blade.

40. A retractor blade according to claim 39 wherein the formation is releasably connected to the blade allowing a user to select working formations according to surgical requirements.
